(19) United States
(12) Patent Application Publication
(10) Pub. No.: US 2026/0198778 A1
(43) Pub. Date: Jul. 16, 2026

(54) SYSTEMS AND METHODS FOR DECODING USER INTENT FROM MULTIMODAL BIOSIGNALS

(71) Applicant: Richard Hull Doss, Los Angeles, CA (US)

(72) Inventors: Albert Frank Shore, Reading, PA (US); Gregory Lynn Gillispie, Poway, CA (US); Milomir Kotlajic, Belgrade (RS); Nenad Radosavljevic, Belgrade (RS); Vladimir Tesanovic, Belgrade (RS); Lazar Ivanovic, Belgrade (RS)

(21) Appl. No.: 19/446,733

(22) Filed: Jan. 12, 2026

Related U.S. Application Data
(60) Provisional application No. 63/744,344, filed on Jan. 12, 2025.

(51) Int. Cl. A61B 5/00; A61B 5/256; A61B 5/291; A61B 5/296; A61B 5/372; A61B 5/384

(52) U.S. Cl. CPC A61B 5/0006; A61B 5/256; A61B 5/291; A61B 5/296; A61B 5/372; A61B 5/384

(57) ABSTRACT

Systems and methods are disclosed for decoding user intent from multimodal biosignals. Around-the-ear electroencephalography (EEG) electrodes and throat sensors acquire signals associated with cortical activity and subvocal muscle activity. A biosignal acquisition device digitizes the EEG and throat biosignals and transmits them to a mobile computing device. The mobile computing device performs signal conditioning, feature extraction, and machine-learning-based classification to generate intent outputs, including at least a binary YES or NO intent. The intent outputs are transmitted to a wearable display device, such as smart glasses, which present visual, audio, or haptic feedback. In certain embodiments, the system connects to a remote server for logging, analytics, adaptive model training, fleet-wide model updates, and relaying intent tokens for telepresence or multi-user communication.

### CROSS-REFERENCE TO RELATED APPLICATIONS

The present application is related to and claims priority to U.S. Provisional Ser. No. 63/744,334, filed Jan. 12, 2025, entitled “Ubiquitous Neural Interface Technology,” the entirety of which is incorporated herein by reference.

### FIELD OF THE INVENTION

The present invention relates generally to brain-computer interfaces and biosignal processing systems, and more particularly to systems and methods for decoding user intent from electroencephalography (EEG) and throat biosignals using machine-learning models and presenting decoded intent via wearable display devices.

### BACKGROUND

Brain-computer interface (BCI) systems aim to translate neural or related physiological signals directly into user intent. Current limitations hinder the widespread use of many conventional EEG-based BCIs, which typically demand dense scalp electrode arrays, controlled laboratory environments, and powerful computing resources, making them impractical for mobile and everyday applications.

Moreover, these systems often rely exclusively on EEG, failing to incorporate valuable complementary data sources such as throat electromyography (EMG), which captures subvocal muscle activity or silent articulation activity, for example laryngeal micro-movements or silent mouthing associated with covert speech.

Therefore, a need exists for compact, mobile, multimodal systems capable of accurately decoding user intentions, including simple binary choices, within real-world settings and delivering decoded intent to standard consumer-grade wearable devices.

### SUMMARY

In one aspect, a system for decoding user intent from biosignals is disclosed. The system integrates around-the-ear EEG electrodes and at least one throat sensor to acquire biosignals. A biosignal acquisition device digitizes these EEG and throat biosignals and transmits them to a mobile computing device. The mobile computing device processes the signals through conditioning and feature extraction, then applies one or more machine-learning models to generate an intent output, which includes at a minimum a binary YES or NO indication of user intent. The final intent output is received, presented, and optionally acted upon by a display device, audio device, or online service.

In another aspect, a computer-implemented method involves acquiring EEG and throat biosignals, performing filtering and feature extraction, applying a trained machine-learning model, and transmitting the decoded intent to a display device, audio device, or online service. In a further aspect, a non-transitory computer-readable medium is provided, storing instructions that, when executed by one or more processors, enable the performance of the method.

### BRIEF DESCRIPTION OF THE DRAWING

FIG. 1 illustrates, in a schematic block diagram, an exemplary system configured to decode user intent from multimodal biosignals and to display the decoded intent using a display device, audio device, or online service.

### DETAILED DESCRIPTION

The embodiments described herein relate to systems, methods, and devices that decode user intent from multimodal biosignals, including EEG and throat biosignals, and that present decoded intent via wearable display devices, audio devices, or online services. Although specific embodiments are described, the disclosed systems and methods may be embodied in many different forms and should not be construed as limited to the examples set forth herein.

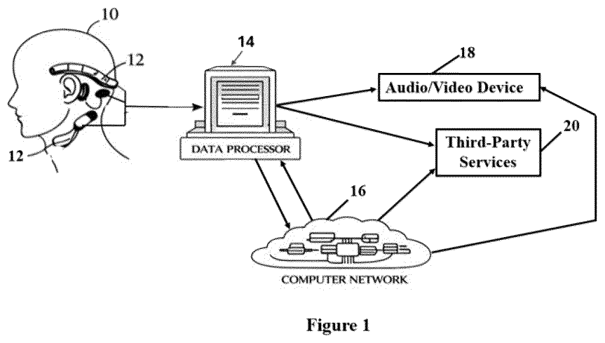

FIG. 1 illustrates, in a schematic block diagram, an exemplary biometric intent-communication system including a user 10 equipped with one or more biometric sensors 12 positioned on the head and adjacent anatomical regions (e.g., around-the-ear, jawline, and/or cervical locations) to acquire multimodal biometric signals. The biometric sensors 12 provide the acquired signals to a data processor 14, which may comprise one or more computing devices configured to execute a processing pipeline including analog front-end conditioning, digitization, and digital preprocessing such as filtering, artifact suppression, normalization, segmentation into time windows, and feature extraction. The data processor 14 further executes intent inference using one or more machine-learning models, including without limitation convolutional neural networks (CNNs), recurrent neural networks (RNNs), and/or correlation or multivariate analysis models such as canonical correlation analysis (CCA), to generate an intent output or control token from the biometric signals. The data processor 14 communicates over a computer network 16 with remote computing resources and third-party services 20, such as messaging/voice services, application programming interfaces (APIs), identity and access services, analytics and telemetry services, and model-update/personalization services. The inferred intent output is delivered to an audio and visual device 18 (e.g., a combined audio/visual output device or one or more coupled output devices) for presentation to the user or for transmission to other devices and services, and in some embodiments the computer network 16 and the third-party services 20 provide feedback data, configuration parameters, and/or updated models to the data processor 14 to improve inference accuracy and system performance.

In certain embodiments, the system may comprise four primary components: an around-the-ear EEG electrode array, at least one throat biosensor, a biosignal acquisition device, and a mobile computing device such as a smartphone. The EEG electrode array and the throat biosensor are implemented as separate, compact, battery-powered sensor devices designed for real-world mobility and wearability. In certain embodiments, the biosignal acquisition device can be integrated with the mobile computing device in either hardware or software.

Both sensor devices may include a low-power wireless interface, preferably Bluetooth 5, for independent communication with the mobile computing device. In such an architecture, the mobile computing device acts as a central device managing simultaneous connections and receiving multiple data streams, while the EEG electrode array device and the throat biosensor device function as peripherals.

Communication between the sensor devices and the mobile computing device may be secured by utilizing protected generic attribute profile (GATT) characteristics to ensure data integrity and user privacy. To enhance durability and water resistance, both sensor devices may incorporate inductive charging capabilities, thereby eliminating the need for exposed charging ports. The mobile phone is responsible for subsequent signal processing, machine-learning inference, and transmission of decoded intent to a wearable display device, audio device, or online service.

In operation, a synthetic telepathy interface captures biosignals using around-the-ear EEG electrodes and throat biosensors and transmits the biosignals to the mobile computing device for processing. A multichannel acquisition module digitizes the biosignals and streams them via Bluetooth to the smartphone. The smartphone executes a signal-processing and machine-learning pipeline that converts raw EEG and throat biosignals into low-bandwidth intent tokens.

Around-the-ear EEG electrodes capture cortical activity related to attention, event-related potentials, steady-state visually evoked potentials, and covert speech. Throat sensors located near laryngeal and submandibular regions capture subvocal muscle activity correlated with internal vocabulary items, such as “yes” or “no.”

Decoded intent tokens are relayed via a short-range wireless protocol to a wearable display device or an audio device, which renders the decoded intent for the user and optionally for remote observers. In some embodiments, output is presented on devices including smart glasses or headphones or sent to a remote service. Decoded intent may be relayed to a cloud service for logging, retraining, telepresence applications, or fleet-wide model updates.

The system acquires two distinct sets of biosignals. A first set, consisting of EEG signals, is captured using around-the-ear sensor arrays. A second set is obtained via a throat sensor, which records surface EMG signals and vibration or audio data.

To digitize these signals, the sensors employ high-sensitivity analog-to-digital converters with a minimum 16-bit resolution. Sampling rates are set at approximately 200 to 1,000 hertz for EEG and EMG channels and approximately 20 hertz to 4 kilohertz for the vibration or audio channel.

The digitized biosignals are transmitted to the mobile computing device using Bluetooth 5. A dedicated smartphone application manages a continuous input buffer for both EEG and throat sensor data and is responsible for time-synchronizing incoming data before segmenting and forwarding the data to subsequent signal-processing stages.

Upon reception, raw EEG signals undergo digital band-pass filtering to isolate frequency content relevant to a specific application. For event-related potential paradigms, a typical passband of approximately 0.1 to 30 hertz may be utilized. For covert speech and steady-state visually evoked potential paradigms, a passband of approximately 5 to 45 hertz may be applied. A notch filter at 50 or 60 hertz may be used to suppress mains interference across all paradigms.

Throat EMG signals may be processed using a high-pass filter in a band such as 20 to 450 hertz to isolate muscular activity, followed by rectification of the signal and low-pass filtering with a cutoff of approximately 5 to 10 hertz to create a smooth EMG envelope representing the magnitude of subvocal contractions.

Processing of audio or vibration signals from the throat sensor may involve acquisition and digitization of the signal, application of a band-pass filter from approximately 20 hertz to 4 kilohertz to isolate relevant content, and extraction of audio features from the filtered signal for inclusion in a feature vector.

In some embodiments, artifact-removal techniques are applied to mitigate noise sources such as eye blinks, jaw clenching, powerline interference, and gross motion. Non-limiting examples of artifact-removal methods include independent component analysis, regression-based artifact subtraction, adaptive filtering such as least-mean-squares or recursive least-squares filters, wavelet denoising, and spatial filtering.

Motion estimates derived from accelerometers, gyroscopes, electrode-impedance monitoring, or high-frequency power metrics may be used to compute an artifact score, which can be used to down-weight or discard highly contaminated segments before feature extraction and decoding.

The preprocessed EEG and EMG signals are routed to an epoching and feature-extraction module. For paradigms time-locked to stimuli, such as binary selection based on event-related potentials, the system defines epochs aligned to stimulus events. Each epoch may encompass approximately negative 200 milliseconds to approximately positive 800 milliseconds relative to stimulus onset, with baseline correction applied using a pre-stimulus interval.

For steady-state visually evoked potential paradigms or continuous covert speech decoding, the system segments data into sliding windows of approximately 0.5 to 2.0 seconds with an overlap in a range of approximately 50 to 75 percent.

From each epoch or window, the system computes EEG features that may include time-domain samples down-sampled to a lower rate, bandpower across one or more frequency bands, frequency-domain components at stimulus frequencies and harmonics, and spatially filtered components. Throat EMG features may include EMG envelope amplitude statistics, short-time energy, zero-crossing rate, and spectral coefficients such as mel-frequency cepstral coefficients. Additional vibration or audio features may include amplitude, frequency-spectrum components, and temporal patterns.

The system constructs feature vectors by concatenating selected EEG and throat features per epoch or window. In certain embodiments, features are normalized or standardized across channels and sessions prior to input into a machine-learning model.

The system employs various machine-learning models for interpreting neural and muscular signals. For binary control, such as YES or NO decisions, a compact convolutional neural network may be utilized, optimized for around-the-ear electrode layouts and configured to process multi-channel EEG, EMG, and vibration data over time to output class probabilities for a label set including at least YES and NO.

In some embodiments, filter-bank canonical correlation analysis supports steady-state visually evoked potential based binary control by correlating EEG, EMG, and vibration signals with a bank of reference sinusoids and harmonics at candidate stimulus frequencies and selecting a class associated with a highest correlation score.

For inner-speech or vocabulary recognition, sequence models such as one-dimensional convolutional networks, recurrent neural networks, gated recurrent units, long short-term memory networks, or transformer-based models may be used to process sequences of EEG, EMG, and vibration features and to output token probabilities for a defined set of internally uttered words or phonemes.

In some configurations, multimodal late fusion is implemented. Separate models for EEG, EMG, and vibration or audio first generate individual intermediate representations, and a fusion layer then combines the representations into a single intent probability distribution. Fusion-layer weights can be adapted to prioritize less noisy input representations.

Models are deployed on the mobile computing device using an on-device inference framework that supports quantization to reduce latency and power consumption.

The system may employ a per-user calibration phase. During calibration the user is presented with labeled prompts, such as YES or NO queries or flashing visual targets. To respond, the user internally repeats a word corresponding to a correct label and may perform covert or subvocal articulation such as silent mouthing or subtle laryngeal activation. These actions produce distinct EEG patterns and, when covert or subvocal articulation is performed, distinct throat-biosignal patterns that are captured by the sensors. The system records multiple trials for each class, extracts features, and trains or fine-tunes one or more classification models.

To reduce data requirements, transfer learning may be used. A core model may be pretrained on external datasets containing EEG and throat biosignals from event-related potential, steady-state visually evoked potential, and covert-speech tasks. User-specific calibration then adapts only a subset of parameters, such as parameters of one or more final dense layers.

During active use, the system can employ online adaptation. Predictions made with high confidence are logged as pseudo-labeled samples, which may be used periodically to re-estimate normalization parameters or to fine-tune classifier weights, thereby compensating for electrode movement, physiological fluctuations, or environmental changes.

During live operation, the smartphone application continually captures and buffers biosignals, segments them into time windows, calculates feature vectors, and inputs the feature vectors into the trained models to obtain class-probability distributions representing user intent. For binary control, selection of YES or NO may be made when a predicted probability surpasses a threshold. For multi-class vocabularies, the highest class probability may be required to exceed a word-specific threshold, otherwise the system may default to a confirm-or-deny mode.

Model outputs are exposed to other applications through an intent application programming interface that provides a compact representation of intent, including a symbolic label and an associated confidence score. Third-party applications, including applications for smart glasses, may subscribe to this intent stream.

The smartphone transmits intent tokens to a wearable display device using a short-range wireless protocol such as Bluetooth. The wearable device decodes and presents the intent to the user as a visual overlay, audio cue, haptic pattern, or notification.

In some embodiments, the mobile computing device forwards intent events to a remote server over an encrypted network connection for purposes such as data logging, remote monitoring, collaborative work, or joint decision-making processes.

As shown in FIG. 1, a user's head 10 is illustrated wearing an around-the-ear EEG electrode array 12 positioned proximate to the ear to capture cortical electrical activity. A biometric sensor 14 is disposed on the user's neck to detect laryngeal and/or subvocal muscle activity and related biomechanical vibrations. The EEG electrode array 12 and biometric sensor 14 are coupled to a biometric sensor acquisition device 16, which amplifies and digitizes corresponding EEG and biometric biosignals. The digitized biosignals are transmitted to a mobile computing device 18, such as a smartphone, which executes a signal-processing, deep-analysis, and intent-decoding stack 22. Within the stack 22, a first stage performs signal preprocessing, a second stage executes deep analysis using one or more convolutional neural networks (CNN), recurrent neural networks (RNN), and canonical correlation analysis (CCA) models, and a third stage performs intent or control-token decoding. The decoded data and control tokens are provided to one or more output devices, including a display device 30, such as smart glasses or another visual display, and optionally an audio device such as headphones. In some embodiments, the mobile computing device 18 further communicates data tokens and related feature summaries to an online service 28 via a ubiquitous compute orchestrator 31. The ubiquitous compute orchestrator 31 coordinates remote analysis, compilation, and aggregation of large-scale biosignal data and returns updated parameters, models, or control policies to the mobile computing device 18 and any paired peripherals, enabling all devices to operate concurrently in a distributed fashion.

As shown in FIG. 2, EEG and biometric signals 40 acquired from the around-the-ear EEG electrode array 12 and biometric sensor 14 are provided to a preprocessing block 42. The preprocessing block 42 performs filtering and artifact removal, including band-pass filtering, notch filtering, and motion-artifact rejection, to produce cleaned signals suitable for decoding. The preprocessed signals are then supplied to a feature-extraction block 44, which computes feature vectors from the EEG and biometric signals, such as band-power measures, time-domain statistics, spectral features, and spatially filtered components. Within the feature-extraction block 44, feature representations are prepared specifically for downstream deep-analysis models including convolutional neural networks (CNN), recurrent neural networks (RNN), and canonical correlation analysis (CCA) models. A deep-analysis block 46 spans from the output of feature extraction through post-processing, and applies the CNN, RNN, and CCA models to generate intermediate probability distributions, confidence metrics, and control tokens. A ubiquitous compute orchestrator, corresponding to element 31 in FIG. 1, may issue a series of ubiquitous-compute calls from the deep-analysis block 46 to remote or peer devices to analyze, extract, and compile large volumes of biosignal data, returning updated weights, thresholds, or fusion rules while the local device continues to operate in real time. The resulting outputs are delivered to a data-output block 48, which aggregates the deep-analysis results and produces a data output that may include binary YES/NO decisions, multi-class vocabulary labels, continuous control variables, or other high-level control tokens suitable for use by wearable displays, audio devices, robotic systems, or remote services.

What is claimed is:

1. A system for decoding user intent from biosignals, comprising:
one or more EEG electrodes configured to be positioned around at least one ear of a user and to generate EEG signals;
one or more throat sensors configured to be positioned proximate to a laryngeal region of the user and to generate throat biosignals;
a biosignal acquisition device coupled to the one or more EEG electrodes and the one or more throat sensors, the biosignal acquisition device configured to digitize the EEG signals and the throat biosignals;
a mobile computing device in communication with the biosignal acquisition device, the mobile computing device configured to receive the digitized EEG signals and throat biosignals, perform signal conditioning on the digitized EEG signals and throat biosignals to generate conditioned signals, extract from the conditioned signals one or more feature vectors comprising EEG features and throat features, and apply at least one machine-learning model to the one or more feature vectors to generate an intent output indicative of a user intent including at least a binary YES or NO intent;
and a wearable display device in communication with the mobile computing device and configured to present the intent output to the user.

2. The system of claim 1, wherein the one or more EEG electrodes comprise an around-the-ear EEG array configured to be positioned bilaterally around both ears of the user.

3. The system of claim 1, wherein at least one throat sensor comprises at least one surface electromyography electrode and at least one vibration or audio sensor adhered to a neck region of the user.

4. The system of claim 1, wherein the biosignal acquisition device comprises a multichannel acquisition board configured to interface with the one or more EEG electrodes and the one or more throat sensors.

5. The system of claim 1, wherein the biosignal acquisition device is configured to digitize each channel of the EEG signals and the throat biosignals at a sampling rate between 200 hertz and 1,000 hertz and with a resolution of at least 16 bits per sample.

6. The system of claim 1, wherein the mobile computing device is configured to receive the digitized EEG signals and throat biosignals via a Bluetooth wireless link.

7. The system of claim 1, wherein the signal conditioning includes applying a first band-pass filter to the EEG signals with a passband in a range of approximately 0.1 to 30 hertz for event-related potential paradigms.

8. The system of claim 1, wherein the signal conditioning includes applying a second band-pass filter to the throat biosignals in a range of approximately 20 to 450 hertz, rectifying the filtered throat biosignals, and low-pass filtering the rectified throat biosignals to obtain an electromyography envelope.

9. The system of claim 1, wherein the mobile computing device is further configured to perform artifact removal by applying independent component analysis to attenuate contributions from eye blinks, jaw clenching, or motion-induced noise in the EEG signals.

10. The system of claim 1, wherein the one or more feature vectors comprise EEG features including at least one of bandpower in one or more frequency bands, time-domain samples, and frequency-domain components at one or more stimulus frequencies and harmonics.

11. The system of claim 1, wherein the one or more feature vectors comprise throat features including at least one of electromyography envelope amplitude statistics, short-time energy, zero-crossing rate, and spectral coefficients.

12. The system of claim 1, wherein at least one machine-learning model comprises a convolutional neural network adapted for around-the-ear EEG inputs.

13. The system of claim 1, wherein at least one machine-learning model comprises a filter-bank canonical correlation analysis model configured to decode steady-state visually evoked potentials.

14. The system of claim 1, wherein at least one machine-learning model comprises a multimodal late fusion network configured to receive a first representation derived from the EEG features and a second representation derived from the throat features and to combine the first and second representations into the intent output.

15. The system of claim 1, wherein the mobile computing device is further configured to generate a confidence score associated with the intent output and to suppress output of the intent when the confidence score is below a predetermined threshold.

16. The system of claim 1, wherein the wearable display device comprises a pair of smart glasses configured to receive the intent output via a short-range wireless connection and to render the intent output as at least one of a visual overlay, an audio cue, or a haptic notification.

17. The system of claim 1, further comprising a remote server configured to receive the intent output over an encrypted network connection for logging, analytics, or retraining of the at least one machine-learning model.

18. The system of claim 1, wherein the mobile computing device is further configured to perform a calibration phase in which labeled examples of user responses are collected and used to train or fine-tune the at least one machine-learning model for a particular user.

19. The system of claim 18, wherein the at least one machine-learning model is pretrained on data from a plurality of users and only a subset of parameters of the at least one machine-learning model is updated during the calibration phase using the labeled examples of the particular user.

20. A method for decoding user intent from biosignals, comprising:
acquiring EEG signals from one or more around-the-ear EEG sensor arrays positioned proximate to at least one ear of a user;
acquiring biosignals from one or more throat sensors positioned proximate to a laryngeal region of the user;
digitizing the EEG signals and the throat biosignals;
filtering the digitized EEG signals with a first band-pass filter and the digitized throat biosignals with a second band-pass filter;
rectifying and low-pass filtering the throat biosignals to obtain an electromyography envelope;
segmenting the filtered EEG signals and the electromyography envelope into time windows;
extracting, from each time window, EEG features and electromyography features to form a feature vector;
inputting the feature vector into a trained machine-learning model that outputs a probability distribution over a plurality of intent classes including at least a YES class and a NO class;
selecting an intent class based on the probability distribution and a confidence threshold; and
communicating an indication of the selected intent class to a user device.

21. The method of claim 20, wherein segmenting the filtered EEG signals comprises defining one or more epochs that are time-locked to stimulus events, each epoch spanning from approximately negative 200 milliseconds to approximately positive 800 milliseconds relative to a stimulus onset and applying baseline correction using a pre-stimulus interval.

22. The method of claim 20, wherein segmenting the filtered EEG signals comprises segmenting the filtered EEG signals into sliding windows of approximately 0.5 to 2.0 seconds with an overlap of approximately 50 to 75 percent for continuous covert speech decoding.

23. The method of claim 20, wherein extracting EEG features includes computing bandpower within one or more predefined frequency bands.

24. The method of claim 20, wherein extracting EEG features includes computing correlation scores between the EEG signals and reference sinusoids corresponding to candidate stimulus frequencies using filter-bank canonical correlation analysis.

25. The method of claim 20, wherein extracting electromyography features includes computing at least one of a mean amplitude of the electromyography envelope, a peak amplitude of the electromyography envelope, short-time energy, or a zero-crossing rate.

26. The method of claim 20, further comprising performing EEG signal artifact removal from the digitized EEG signals using independent component analysis to reduce contributions from eye blinks or muscle activity.

27. The method of claim 20, further comprising, during a calibration phase, presenting labeled prompts to the user, recording corresponding EEG and throat biosignals, and tuning the trained machine-learning model using feature vectors derived from the recorded biosignals and associated labels.

28. The method of claim 27, wherein tuning the trained machine-learning model comprises updating only a subset of parameters of a pretrained model using the feature vectors derived from the recorded biosignals and associated labels.

29. The method of claim 20, further comprising, during live operation, logging time windows associated with high-confidence predictions and updating at least one normalization parameter or model parameter based on the logged time windows to adapt the trained machine-learning model over time.
